(19) United States
(12) Patent Application Publication
(10) Pub. No.: US 2026/0108178 A1
(43) Pub. Date: Apr. 23, 2026

(54) GAIT ANALYSIS AND GAIT GUIDE METHOD DEVICE THROUGH SOUND

(71) Applicant: Dae Geun JEON, Seoul (KR)

(72) Inventor: Dae Geun JEON, Seoul (KR)

(21) Appl. No.: 19/318,960

(22) Filed: Sep. 4, 2025

(30) Foreign Application Priority Data
Oct. 21, 2024 (KR) 10-2024-0143962

(51) Int. Cl. A61B 5/11; A61B 5/00; A63B 24/00

(52) U.S. Cl. CPC A61B 5/112; A61B 5/1126; A61B 5/743; A61B 5/7475; A63B 24/0075; A61B 2562/0204

(57) ABSTRACT

Provided is a gait analysis device and method. The gait analysis device may include an input unit that receives a user's gait sound data, and a gait analysis unit that derives the user's current gait information by analyzing the gait sound data.

### CROSS-REFERENCE TO RELATED APPLICATION

This application claims priority to and the benefit of Korean Patent Application No. 10-2024-0143962, filed on Oct. 21, 2024, the disclosure of which is incorporated herein by reference in its entirety.

### BACKGROUND

### 1. Field of the Invention

The present invention relates to a device and method for analyzing gait and providing gait guidance through sound (gait analysis device and method). In particular, the present invention relates to a gait analysis device and method capable of acquiring gait sound data recorded from a user's gait using a smartphone or other equipment, performing analysis of the user's gait based on the acquired gait sound data, and providing a customized gait-related exercise guide information to the user based on the analysis results.

### 2. Discussion of Related Art

Walking plays an important role in human health and daily life. If one does not walk properly, it may lead to issues such as strain on the spine or fatigue in the feet, making it difficult to walk for extended periods. As a result, there has been ongoing development of technologies such as gait analysis technology and gait guidance technology aimed at guiding or correcting users toward proper walking posture.

However, most of the previously disclosed technologies require dedicated measurement devices for detecting gait (e.g., requiring users to wear inertial measurement units (IMUs) on both feet to perform gait analysis) or are limited to performing gait analysis on patients with specific conditions (e.g., Parkinson's disease). As a result, it is impossible to analyze gait of users who do not have such dedicated measurement equipment, and there is a problem in providing analysis and guidance for gait across diverse populations, including healthy adults, healthy children, athletes, hemiplegic patients, or cerebral palsy patients.

The technology underlying the present invention is disclosed in Korean Patent Publication No. 10-2008-0094172.

### SUMMARY OF THE INVENTION

The present invention is directed to address the issues of the conventional technology described above by providing a gait analysis device and method using sound to solve problems such as inefficient or incorrect walking, which could not be addressed due to the lack of dedicated gait detection measurement equipment.

In particular, the present invention is directed to provide a sound-based gait analysis device and method that may provide sound-based gait analysis and guidance to not only patients with specific conditions (such as leg paralysis, Parkinson's disease, cerebral palsy, etc.) but also various general users (such as adults, children, athletes, etc.), thereby enabling easy access to gait analysis for users.

However, the technical problems that embodiments of the present invention seek to solve are not limited to the technical problems described above, and other technical problems may exist.

As technical means for achieving the aforementioned technical tasks, a gait analysis device according to an embodiment of the present invention may include an input unit that is configured to receive a gait sound data of a user, and a gait analysis unit that is configured to derive a current gait information of the user through analysis of the gait sound data.

Additionally, the gait analysis device according to an embodiment of the present invention may further include a recording control unit that is configured to control the recording of sounds during the user's walking, and the input unit may be configured to receive the recorded gait sound data from the recording control unit as the gait sound data.

Furthermore, the recording control unit may be configured to control the recording process using a user terminal carried by the user.

Furthermore, the gait analysis unit may be configured to derive the gait information by comparing a user gait pattern information derived from the analysis of the gait sound data with a reference gait pattern information predefined through an analysis of gait sound data of a plurality of experimenters previously collected.

Furthermore, the gait analysis device according to an embodiment of the present invention may further include a guide unit that is configured to generate and provides a gait-related exercise guide information based on the gait information.

Meanwhile, the gait analysis method according to an embodiment of the present invention may include a step of receiving a gait sound data of a user in an input unit, and a step of deriving a gait information of the user by analyzing the gait sound data in a gait analysis unit.

The above-described means for solving the problems are merely illustrative and should not be interpreted as limiting the scope of the invention. Additional embodiments may exist in addition to the illustrative embodiments described above, as well as in the drawings and the detailed description of the invention.

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIG. 1 is a diagram illustrating the configuration of a gait analysis system including a gait analysis device according to an embodiment of the present invention,

FIG. 2 to FIG. 6 are drawings illustrating a gait analysis device according to an embodiment of the present invention, and

FIG. 7 is a flowchart illustrating an operation of a gait analysis method according to an embodiment of the present invention.

### DETAILED DESCRIPTION OF EXEMPLARY EMBODIMENTS

Hereinafter, with reference to the drawings, exemplary embodiments of the present invention are described in detail so that a person skilled in the art to which the present invention belongs may easily implement the present invention. However, the present invention may be implemented in various forms and is not limited to the embodiments described herein. Furthermore, in order to clearly describe the present invention in the drawings, parts unrelated to the description have been omitted, and similar parts throughout the specification are indicated by similar reference numerals.

Throughout the specification of the present invention, when a part is said to be “connected” to another part, this includes not only cases where they are “directly connected,” but also cases where they are “electrically connected” or “indirectly connected” through an intermediate element.

Throughout the entire specification of the present invention, when a component is said to be located “on,” “above,” “on top of,” “below,” “under,” or “at the bottom of” another component, this includes not only cases where the component is in contact with another component but also cases where another component exists between the two components.

Throughout the specification of the present invention, when a part is described as “including” a component, this means that it may include other components unless otherwise specified.

Throughout the specification of the present invention, some of the operations or functions described as being performed by a terminal, device, or apparatus may instead be performed by a server connected to the terminal, device, or apparatus. Similarly, some of the operations or functions described as being performed by a server may instead be performed by a terminal, device, or apparatus connected to the server.

Throughout the specification of the present invention, the term “at least one” may be defined as including both singular and plural forms, and it is understood that even if the term “at least one” does not exist, each component may exist in singular or plural form and may mean singular or plural. Additionally, the provision of each component in singular or plural form may be subject to change depending on embodiments.

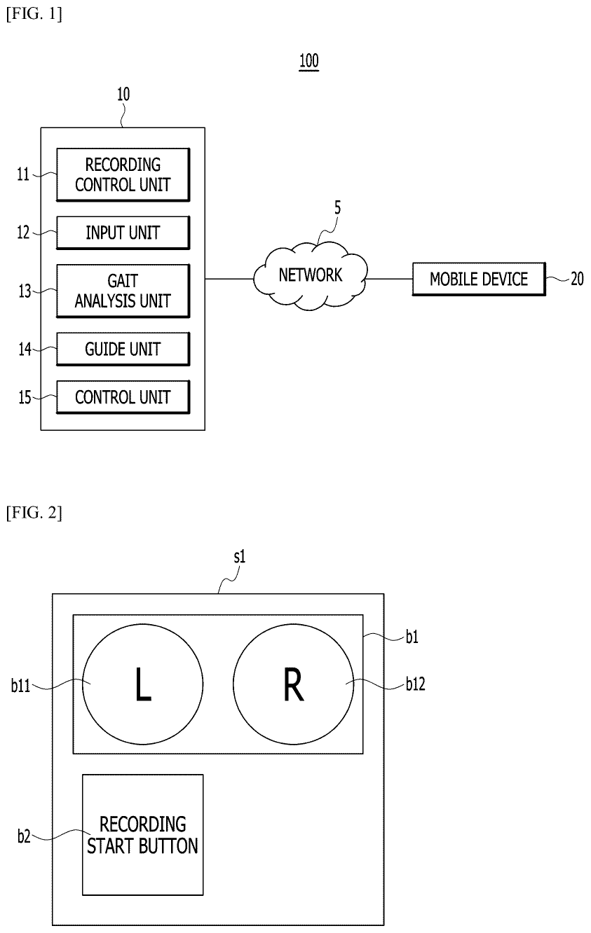

FIG. 1 is a schematic diagram illustrating the configuration of a gait analysis system 100 including a gait analysis device 10 according to an embodiment of the present invention.

For the sake of convenience in the following description, the gait analysis device 10 according to an embodiment of the present invention will be referred to as the device 10, and the gait analysis system 100 according to an embodiment of the present invention will be referred to as the system 100. Additionally, any matters shown in the drawings of the present invention, including FIG. 1, may be omitted in the following description, but the same applies to the description of the device 10.

Referring to FIG. 1, the system (100) may include the device 10 and a user terminal 20.

The device 10 may be a device that analyzes gait through sound and performs gait guidance, and may be referred to as a gait analysis and guidance device through sound, a gait analysis device, etc. The device 10 may be a gait analysis device capable of performing gait analysis through sound and providing exercise guidance. The device 10 may be a device or server that provides at least one of a webpage (homepage), a program, an application (app), a service, or a platform related to gait analysis. In this context, the webpages (homepage), programs, applications, services, and platforms related to gait analysis provided by the device 10 may be referred to as the “webpage (homepage),” “program,” “app,” “service,” and “platform,” respectively, for the sake of convenience in the following description.

In the description referring to FIG. 1, for example, the device 10 is described as being configured in the form of a server capable of transmitting and receiving data with a user terminal 20 via a network 5. That is, in FIG. 1, it may be seen that the device 10 is configured in the form of a server and is separated from the user terminal 20. This is merely one example to aid in understanding the present invention and is not limited thereto. As another example, if the device 10 is implemented in the form of the present program or app and installed on the user terminal 20, the device 10 may be included within the user terminal 20.

The user terminal 20 may refer to the terminal device held by the user who uses the device 10. The user is an individual who wishes to analyze their walking through the device 10 and receive gait-related exercise guidance information based on their current gait information (walking status), and may be anyone, regardless of age or gender. Users may include various types (multiple types) of users, such as children, adults, the elderly, athletes, and patients with specific diseases (e.g., patients with hemiplegia, paraplegia, Parkinson's disease, etc.). In other words, a user may be at least one type of user among the aforementioned multiple types of users.

Users may use the service without installing the program or app on their user device 20, or they may access the program or app on their user device 20 and use the service with or without registering (logging in).

The user terminal 20 may include, for example, a PCS (Personal Communication System), GSM (Global System for Mobile communication), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (Wideband Code Division Multiple Access), Wibro (Wireless Broadband Internet) terminals, smartphones, smart pads, tablet PCs, laptops, wearable devices, and other types of wired and wireless communication devices, and is not limited to these. More preferably, the user terminal 20 considered in the present invention may be a portable smartphone having a recording function (i.e., capable of recording). Therefore, the following description will be based on the example where the user terminal 20 is a smartphone having a recording function.

Additionally, FIG. 1 illustrates an example where the system 100 includes a single user terminal 20, but this is merely one example to aid in understanding the present invention and is not limited thereto. As another example, the system 100 may include multiple user terminals held by multiple users, and the descriptions regarding the user terminal 20 in the following explanation of the present invention may also apply to each of the multiple user terminals, even if such descriptions are omitted.

Additionally, although not shown in the figures, the system 100 may include an administrator terminal (not shown) held by an administrator. The administrator may be the person who develops and distributes the device 10 and operates and manages the device 10. Here, the administrator terminal may include all types of wired and wireless communication devices described above.

The device 10 may be interconnected with each of the user terminals 20 and administrator terminals (not shown) via the network 5 to transmit and receive data, and to control the operation (e.g., screen display activation, etc.) of each of the user terminals 20 and administrator terminals (not shown).

The network 5 may include, for example, a 3GPP (3rd Generation Partnership Project) network, an LTE (Long Term Evolution) network, a WIMAX (World Interoperability for Microwave Access) network, the Internet, a LAN (Local Area Network), a Wireless LAN (Wireless Local Area Network), a WAN (Wide Area Network), a PAN (Personal Area Network), Bluetooth network, NFC (Near Field Communication) network, satellite broadcasting network, analog broadcasting network, DMB (Digital Multimedia Broadcasting) network, etc., but is not limited to these, and may include various wired and wireless communication networks. A more detailed description of the device 10 is as follows.

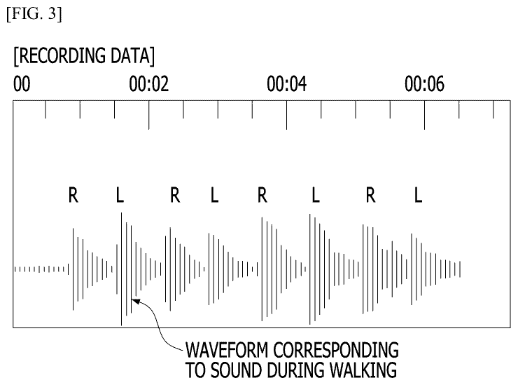

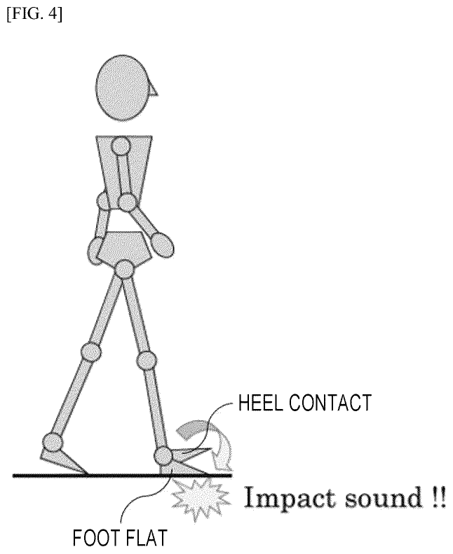

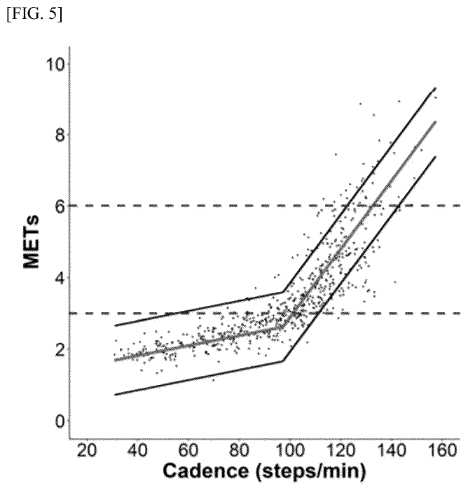

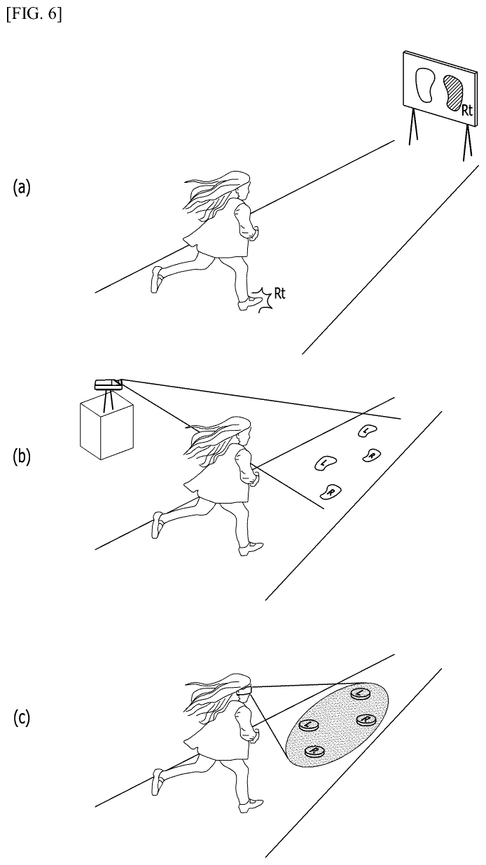

FIG. 2 to FIG. 6 are drawings illustrating a gait analysis device 10 according to an embodiment of the present invention. In particular, FIG. 2 is a drawing illustrating an example of a recording progress screen considered in the device 10, FIG. 3 is a drawing illustrating an example of gait sound data received by an input unit 12 in the device 10, FIG. 4 is a diagram illustrating the sounds generated during walking considered in the device 10, FIG. 5 is a diagram showing the relationship between walking rate and metabolic equivalent considered in the device 10, and FIG. 6 is a diagram illustrating an example of the form in which gait-related exercise guide information is provided in the device 10. The matters described in FIG. 2 to FIG. 6 may be omitted herein, but the same applies to the description of the device 10.

For reference, the figures shown in FIG. 4 of the present invention is excerpted from [Kaoru Abe (2016). Gait analysis through sound, Niigata Journal of Health and Welfare Vol. 15, No. 1], and the figure shown in FIG. 5 of the present invention is excerpted from [Catrine Tudor-Locke (2020). Walking cadence (steps/min) and intensity in 41 to 60-year-old adults: the CADENCE adults study, International Journal of Behavioral Nutrition and Physical Activity].

Referring to FIG. 1 through FIG. 6, the present device 10 may include a recording control unit 11, an input unit 12, a gait analysis unit 13, a guide unit 14, and a control unit 15.

The recording control unit 11 may be configured for the user to control the recording of sounds made by the user during walking. Specifically, the recording control unit 11 may be configured for the user to control the recording of sounds made by the user during walking using the user terminal 20.

Specifically, the recording control unit 11 may display a recording progress screen s1, such as that shown in FIG. 2, on the screen of the user terminal 20 when the user accesses the app (or program) using the user terminal 20.

The recording progress screen s1 may include a start foot selection button b1 for the user to select information about the first starting foot (i.e., the first foot to be stepped) when recording the sound while walking, and a recording start button b2. Here, the start foot selection button b1 may include a left button b11 for selection when the first step is the left foot, and a right button b12 for selection when the first step is the right foot.

The recording control unit 11 may be configured such that, after the recording progress screen s1 is displayed on the user terminal 20, if the user wishes to start the first step of walking with the left foot during recording, guide the user to select the left button b11, and if the user wishes to start the first step of walking with the right foot during recording, guide the user to select the right button b12.

Thereafter, when the recording control unit 11 detects that the user has made a selection of either the left button b11 or the right button b12, the recording control unit 11 may guide the user as follows: after the user clicks the recording start button b2 and holds the user terminal 20 in the user's hand, the user is instructed to walk for a predetermined period of time (for example, 10 seconds) while alternately placing both feet (i.e., including the left foot and the right foot) on the ground. In performing the walking, the user is guided (or prompted) to start walking with the foot corresponding to the button preselected by the user from among the left button b11 and the right button b12 (for example, when the right button b12 is selected, the right foot is used as the initial stepping foot).

In this case, the predetermined period of time may be set to, for example, 10 seconds, and may be pre-set by the administrator. The setting of the predetermined period of time may be set and changed to various values by the administrator.

Additionally, the recording control unit 11 may control the recording to be performed in conjunction with a microphone or other electronic device adjacent to the user's foot or the ground.

Furthermore, the recording control unit 11 may control the recording so that, when the user clicks the recording start button b2, control the operation of the user terminal 20 such that waveform data corresponding to the gait sounds generated by the user's walking (e.g., recording signal waveform data as shown in FIG. 3) recorded through the recording device of the user terminal 20 during the predetermined time period is displayed in real time on the screen of the user terminal 20.

The recording control unit 11, while recording gait sounds as described above, may display, in real time, waveform data corresponding to the gait sounds on the screen of the user terminal 20. This allows the user to confirm, during walking, whether a waveform corresponding to the sound generated by the right foot contacting the ground (i.e., a right-foot corresponding waveform) is immediately displayed on the screen of the user terminal 20 when, for example, the user's right foot makes contact with the ground, and whether, thereafter, a waveform corresponding to the sound generated by the left foot contacting the ground (i.e., a left-foot corresponding waveform) is immediately displayed on the screen of the user terminal 20 when the user switches feet and the left foot makes contact with the ground. That is, the recording control unit 11 may, while recording, display the waveform data in real time on the screen of the user terminal 20, thereby enabling the user to intuitively confirm, in real time, whether the sounds generated by the foot contacting the ground at each alternation of the left and right feet during walking are being properly recorded.

Additionally, in the waveform data (recording data) shown in FIG. 3, the horizontal axis represents time, and the vertical axis represents sound intensity (decibels). The waveform data of the gait sounds recorded when the user alternates their feet while walking may be represented as a waveform where the recorded (measured) sound intensity increases and decreases repeatedly in sync with the alternation of the user's feet, as shown in FIG. 3.

In consideration of the above, when displaying the waveform data in real time on the screen of the user terminal 20, the recording control unit 11 may display, on the real-time waveform data, a mark “R” indicating a right-foot corresponding waveform and a mark “L” indicating a left-foot corresponding waveform, as illustrated in FIG. 3, so that the user may more intuitively distinguish between the right-foot corresponding waveform and the left-foot corresponding waveform in the real-time displayed waveform data and thereby confirm whether recording is being properly performed. In this case, for the first peak point at which the largest sound is detected for the first time in the real-time waveform data (i.e., waveform data in the form of amplitude repeatedly increasing and decreasing), the recording control unit 11 may display the mark “R” corresponding to the foot in the button preselected by the user from among the left button b11 and the right button b12 (for example, when the right button b12 is selected, the right foot). Thereafter, whenever peak points repeatedly appear, the marks “L” and “R” may be alternately displayed in real time at the positions of the respective peak points. By providing such real-time marking, the recording control unit 11 may enable the user to intuitively confirm, for example, when the right foot actually makes contact with the ground, whether the sound generated by such contact of the right foot has been properly recorded, through the screen.

Thereafter, the recording control unit 11 may automatically suspend the recording process after a predetermined time has elapsed following the user's click on the recording start button b2 (i.e., after a predetermined time has passed from the moment the recording start button is clicked), automatically stop the recording process, and subsequently display the recording data corresponding to the user's gait sounds recorded (completed) during the predetermined time period on the screen of the user terminal 20 in response to the stop. In this case, the recording data may include waveform data corresponding to the user's gait sounds recorded during the specified time period as the user walks, and may be the same as shown in FIG. 3. Accordingly, the recording data recorded by the recording control unit 11 may include sounds when the right foot touches the ground (right foot sounds) and sounds when the left foot touches the ground (left foot sounds) in a distinguishable state.

In this case, the recording control unit 11 may display a save button and a re-record button together in a specific area of the screen of the user terminal 20 when displaying the recording data on the screen. Here, the recording control unit 11 may guide the user to confirm the recording data, and if the confirmation result shows that if the waveforms of the left foot and right foot in the recording data are properly distinguished and displayed, the recording control unit 11 may guide the user to click the save button, thereby enabling the recording data to be stored in a database unit (not shown) of the device 10 through the click of the save button. Additionally, the recording control unit 11 may, if the confirmation results indicate that the waveforms of the left foot and right foot in the recording data are not distinguished normally and clearly, prompt the user to click the “re-record” button, thereby enabling re-recording of the sounds during walking through the click on the “re-record” button.

Additionally, while walking involves multiple stages, it is generally known that the sounds produced during walking typically occur only at two specific points, as shown in FIG. 4: 1) when the heel of the foot makes contact with the ground (Heel Contact), and 2) when the entire sole of the foot makes contact with the ground (Foot Flat). Accordingly, the recording data may include (record) the sounds generated during heel contact and foot flat contact each time the user alternates between walking with both feet, i.e., each time the left foot and right foot come into contact with the ground. In this case, in the present invention, it is desirable that the heel contact sound and the foot flat sound be recorded in a manner that distinguishes them from each other. However, in various cases such as when the user is walking quickly or when the ankle is stiff, these two types of sounds may not be clearly distinguished and recorded. Accordingly, in the present invention, it does not matter (i.e., it is irrelevant) even if these two types of sounds (i.e., heel contact sound and foot flat sound) are recorded without being distinguished from each other.

The device 10 analyzes the recorded data to derive the user's current gait information and generates gait-related exercise guide information based on this information, which is provided to the user to guide the user to perform walking with a better posture and in a more comfortable manner.

Accordingly, the recording control unit 11 may cause the user to record the sounds made by the user while walking using a personal smartphone, i.e., the user terminal 20. In addition, the recording control unit 11 may be configured for the user to control the recording so that, when necessary, the user may record sounds using a microphone close to the ground and feet or a microphone of other electronic equipment in conjunction with the smartphone (the user terminal carried by the user).

That is, the recording control unit 11 may be configured for the user to control the recording of the gait sounds of the user using the user terminal 20 held by the user, and, in particular, may be configured for the user to control the recording to be performed with the user terminal 20 positioned as close to the foot as possible during recording. In addition, when the user is about to start recording the gait sounds, the recording control unit 11 may be configured for the user to preselect, prior to commencement of the recording, information on which foot among the two feet will first make contact with the ground (i.e., information on the initial stepping foot).

The input unit 12 may receive the user's gait sound data. The input unit 12 may receive the recording data of the user's gait sound (in particular, the recording data recorded using the user terminal 20) recorded through the recording control unit 11 as the gait sound data. In other words, the input unit 12 may receive the recorded data stored by the user clicking the save button as the user's gait sound data.

Accordingly, the gait sound data input to the input unit 12 may be recording data of the user's gait (walking) sounds recorded using the user terminal 20, and may be the same as that shown in FIG. 3.

In this case, the gait sound data is shown as recording data of the sound made while walking, but this is just an example to help understanding the invention and is not limited thereto. The gait sound data may include not only recording data of the sounds (footsteps) made during walking but also recording data of the sounds (footsteps) made during running.

In other words, the recording control unit 11 not only controls the recording of sounds made while walking, but also controls the recording of sounds made while running after completing the recording of sounds made while walking. In this case, the recording control unit 11 may measure the user's heart rate using a wearable device worn on a part of the user's body while recording the sound during running. In other words, the recording control unit 11 controls the recording of the sound during running in the same manner as the recording of the sound during walking, and at the same time, measures the user's heart rate using other devices such as a wearable device.

Additionally, the recording control unit 11 may control the recording of sounds during running by multiple experimenters, as described below, and control the measurement of heart rate during running, in order to enable a derivation of the user's current gait information by the gait analysis unit 13 described below. The recording control unit 11 may enable the gait analysis unit 13, which will be described later, to derive the user's gait information based on the recorded data of sounds during walking obtained from the user and multiple experimenters, and the recorded data of sounds during running obtained from the user and multiple experimenters (especially the recorded data of sounds during running obtained in conjunction with heart rate). In this case, the recording data of sounds during running may be acquired from among multiple experimenters, particularly healthy adults (i.e., normal experimenters), or may be acquired from all experimenters, including not only normal individuals but also patients.

The gait analysis unit 13 may derive the user's current gait information (walking state information) by analyzing the gait sound data input in the input unit 12.

The gait analysis unit 13 may perform analysis (measurement) of the user's gait characteristic information based on the gait sound data, and derive the user's current gait information based on the analyzed gait characteristic information.

Here, the gait characteristic information may include a first characteristic information corresponding to the foot contact sound intensity (in decibels), a second characteristic information corresponding to the foot contact duration (in milliseconds, in seconds), a third characteristic information corresponding to the frequency of the right foot and the left foot and the cadence of both feet, and a fourth characteristic information corresponding to the interval between the foot contacts of both feet.

That is, the third characteristic information may include characteristic information corresponding to the frequency of the right foot, characteristic information corresponding to the frequency of the left foot, and characteristic information corresponding to the frequency of both feet. Here, the frequency of the right foot represents the number of times a sound is generated by the right foot during a specified time period (e.g., 10 seconds) due to the right foot touching the ground, and similarly, the frequency of the left foot represents the number of times a sound is generated by the left foot during the same time period due to the left foot touching the ground. Additionally, the characteristic information corresponding to the frequency of both feet among the third characteristic information may represent the walking rate (Cadence), where the walking rate may represent the number of steps (i.e., the total number of times the feet touches the ground) per unit time (e.g., per minute or per the aforementioned 10 seconds). The number of steps may also be referred to as the number of strides.

Additionally, the fourth characteristic information may correspond to the interval between the two feet touching the ground, which may include information on the interval time from the right foot to the left foot and the interval time from the left foot to the right foot.

The gait analysis unit 13 may analyze (measure) the user's gait characteristic information based on the gait sound data, derive a user gait pattern information using the analyzed gait characteristic information, and then compare the user gait pattern information derived from the analysis of the gait sound data with a reference gait pattern information predefined through the analysis of the gait sound data of multiple experimenters previously collected, thereby deriving the user's current gait information (gait status information). Here, the user gait pattern information may refer to pattern information corresponding to the analyzed gait characteristic information.

In this case, the gait analysis unit 13 may, when deriving the user's gait information, take into consideration the user gait pattern information, the user's physical characteristic information previously input by the user, and information indicating whether the user is a patient, so as to derive the gait information. Here, the user's physical characteristic information may include information regarding the user's age, height, weight, and the length of each of the left and right legs (i.e., the left leg length and the right leg length). Here, the information regarding the length of each of the left and right legs may be omitted if it is impossible for the user to measure and input the same.

Specifically, the gait analysis unit 13 may collect experimenter data from multiple experimenters and apply the collected experimenter data as input to an artificial intelligence model for training in order to derive the user's gait information.

In this case, the experimenter data may be data that is composed of (or includes) experimenter physical characteristic information, which is physical characteristic information of the experimenter, gait sound data of the experimenter, and gait pattern information of the experimenter (which may mean pattern information corresponding to gait characteristic information of the experimenter analyzed from the gait sound data of the experimenter), which are linked together as a set. Additionally, the experimenter data may further include information, input by the experimenter, on whether the experimenter is a patient and, if so, the type of disease the experimenter has. Here, the patient status information may indicate that the experimenter is labeled as a “patient” if the experimenter has at least one disease, and as a “non-patient” if the experimenter does not have at least one disease. The information on the type of disease may include, for example, left hemiplegia, right hemiplegia, paraplegia, Parkinson's disease, etc., but is not limited to these, and may include various diseases. Additionally, each of the multiple experimenters may be a user using the device 10, or may be a recruiter separately recruited by an administrator.

The gait analysis unit 13 may train the artificial intelligence model to derive at least one reference gait pattern information for each physical type (i.e., age, height, weight, left leg length, and right leg length) from the physical characteristic information, using the experimenter data of the plural experimenters as input values. Here, at least one physical type may refer to each of the multiple physical types, or a combination of two or more of the multiple physical types. Additionally, the reference gait pattern information may refer to an average gait pattern information derived by averaging the gait pattern information of a subset of multiple experimenters having the same physical type (e.g., a subset of 100 experimenters with the same age, height, and weight).

Furthermore, the gait analysis unit 13 may classify the experimenter data of multiple experimenters into a normal group (experimenter data of normal individuals) and a patient group (experimenter data of patients) when training the artificial intelligence model, and then train the model separately for each group. The gait analysis unit 13 may use the pre-trained artificial intelligence model to derive and predefine the at least one physical type-specific reference gait pattern information for each of the normal group and patient group, and store the defined information in the database unit (not shown) within the device 10.

Here, the artificial intelligence (AI) model may refer to, for example, deep learning models, machine learning models, neural network models (artificial neural network models), neuro-fuzzy models, and may include, for example, convolution neural networks (CNN), recurrent neural networks (RNN), deep neural networks, and other neural network models that have been previously disclosed or may be developed in the future.

Therefore, the gait analysis unit 13 may derive the user's current gait information by comparing the user gait pattern information with the predefined reference gait pattern information (specifically, the reference gait pattern information for each physical type for both the normal group and the patient group) using the artificial intelligence model. Additionally, the gait analysis unit 13 may apply the user's physical characteristic information, previously input by the user, and the user's patient status information (for example, a normal status) to an input value of a pre-trained artificial intelligence model. As an output value corresponding to the input value from the artificial intelligence model, the gait analysis unit 13 may obtain reference gait pattern information matching the input value (i.e., reference gait pattern information corresponding to an average gait pattern information derived by averaging gait pattern information of a plurality of experimenters having the same physical characteristic information as the user's physical characteristic information and having a patient status corresponding to normal). Thereafter, by comparing the reference gait pattern information obtained from the artificial intelligence model with the user's gait pattern information, the gait analysis unit 13 may derive the user's gait information. By way of example, suppose that the user's physical characteristic information is [age: 30 years, height: 157 cm, weight: 50 kg, left leg length and right leg length: 80 cm each], and the user's patient status information is “normal,” and that, by applying this information as an input value to the artificial intelligence model, the obtained reference gait pattern information is [for certain normal experimenters having an age of 30 years, height of 157 cm, weight of 50 kg, and left and right leg lengths of 80 cm each, the reference gait pattern information is “the contact duration of both feet is 0.5 seconds each, and the foot contact interval between the two feet is 20 cm”]. Further, suppose that the user gait pattern information is [with respect to the “contact duration of both feet,” the contact duration of the left foot is 0.3 seconds and the contact duration of the right foot is 0.5 seconds, and with respect to the “foot contact interval between both feet,” the interval from the left foot to the right foot is 15 cm, and the interval from the right foot to the left foot is 20 cm].

In such a case, the gait analysis unit 13 may, through comparison of the reference gait pattern information and the user gait pattern information, derive the user's current gait information (gait status information), for example, as follows: [Compared to normal experimenters, the user has a contact duration of both feet in which the left foot is 0.3 seconds and the right foot is 0.5 seconds, which are different from each other. In particular, the contact duration of the left foot is shorter by 0.2 seconds compared to the average contact duration (0.5 seconds). In addition, the foot contact interval between both feet is also different between the transition from the left foot to the right foot and the transition from the right foot to the left foot. In particular, the foot contact interval during the transition from the left foot to the right foot is shorter by 5 cm compared to the average interval (for example, 20 cm, which is the average interval during the transition from the left foot to the right foot among experimenters)].

The guide unit 14 may generate gait-related exercise guide information based on the current gait information (gait status information) derived from the gait analysis unit 13 and provide it to the user terminal 20. In this case, the gait-related exercise guide information may be gait-related exercise guide information that guides the user's current gait pattern information to change into a gait pattern (normal gait pattern) corresponding to the reference gait pattern information (especially the reference gait pattern information related to normal experimenters) (i.e., guiding the user to walk normally with a normal gait pattern).

Additionally, the guide unit 14 may provide the gait-related exercise guidance information to the user terminal 20 by audibly presenting (providing) the gait-related exercise guidance information through a speaker of the user terminal 20 or through headphones carried by the user (e.g., wireless headphones), using a metronome method. In other words, the guide unit 14 may provide the gait-related exercise guidance information through metronome-style auditory guidance, thereby guiding the user to walk normally in accordance with the metronome-style auditory guidance.

A metronome is a tool used in music to maintain or practice a steady tempo (beat), and is typically a mechanical or electronic device that provides a steady beat at set intervals through sound or visual signals. A metronome may set the speed in BPM (Beats Per Minute), for example, setting it to 60 BPM will produce 60 beats per minute. The guide unit 14 may provide gait-related exercise guidance information through this metronome-style auditory guidance, enabling the user to walk in time with the beats provided by the metronome. This helps correct the user's current gait pattern (abnormal walking) and enables the user to walk normally.

### Example of Providing Gait-related Exercise Guidance Information for Normal Individuals

The guide unit 14 may provide the following gait-related exercise guide information (e.g., the first guide information to the fourth guide information described below) to users who are normal. Here, gait-related exercise guide information may be referred to as “guide information” for convenience in the following description. 1) If the left leg of a user is relatively shorter than the right leg, the shorter leg (e.g., the left leg) tends to produce a louder sound during walking compared to the longer leg and has a higher frequency (number of times the foot touches the ground). In consideration of the foregoing, the guide unit 14, when the user's current gait information derived by the gait analysis unit 13 is, for example, [the sound level of the user's left foot contact is 40 decibels (dB), and the sound level of the user's right foot contact is 50 decibels], may determine that “the sound of the left foot is lower in volume than that of the right foot due to the left leg being shorter than the right leg.” Thereafter, based on the determined information, as the guide information (i.e., gait-related exercise guide information) for inducing normal gait, the guide unit 14 may provide to the user terminal 20 a first guide information relating to short-leg stretching intended to lengthen the shorter leg. That is, the first guide information may include a video (video content), image, or the like related to short-leg stretching for inducing the lengthening of the shorter leg.

2) Additionally, if there is an issue with one of the user's feet, as mentioned earlier, there is a tendency for the duration of foot contact or the interval between foot contacts to differ between the two sides. Of course, this may vary between normal individuals and patients, but generally, if the left foot is painful, the duration of foot contact on the left foot tends to be relatively shorter than that on the right foot, and the interval from the left foot to the right foot tends to be relatively shorter than the interval from the right foot to the left foot.

In consideration of the foregoing, when the user's current gait information derived by the gait analysis unit 13 is, for example, [the contact duration of the left foot is 0.3 seconds and the contact duration of the right foot is 0.5 seconds], the guide unit 14 may provide to the user terminal 20 a second guide information relating to a gait guide for inducing gait (step) correction, so that the user can perform gait in such a manner that the foot (for example, the left foot) having a shorter contact duration among the two feet has the same contact duration as the other foot (for example, the right foot), as the guide information.

In this case, when providing the second guide information, the guide unit 14 may display, on the screen of the user terminal 20, in a pop-up format, a pain presence inquiry message for inquiring whether the left foot (or left leg), which is the foot having the shorter contact duration, is experiencing pain. Thereafter, when the user responds to the inquiry message with “pain present,” the guide unit 14 may provide to the user terminal 20 a message such as “It is recommended that you seek medical consultation.” If the user responds to the inquiry message with “no pain,” the guide unit 14 may provide to the user terminal 20 a video content or the like related to stretching and strengthening training for the left foot (or left leg) (for example, muscle strengthening training for the left leg). A database unit (not shown) within the present device 10 may include a plurality of video contents pre-registered by an administrator, and at the time of providing gait-related exercise guide information, at least one of the plurality of video contents may be provided.

Additionally, the guide unit 14 may provide the aforementioned second guide information related to gait guidance to the user terminal 20 in such a way that the gait guidance is provided audibly based on the metronome method (i.e., in the form of rhythmic music/beat/tempo sounds) through the user's smartphone (i.e., the user terminal 20) or the headphones the user is wearing, as previously described. The guide unit 14 may guide the user to step in time with the provided regular rhythm by providing the gait guide audibly based on the metronome method in this way. By way of example, when providing the gait guide, the guide unit 14 may, in a case where the sound of the right foot is denoted as “thump” and the sound of the left foot is denoted as “tap,” audibly provide the “thump” and “tap” sounds in a recommended rhythm and for a recommended duration (that is, for example, audibly provide the “thump” and “tap” sounds repeatedly at regular intervals of 0.5 seconds), and, in such a case, may additionally provide music (recommended music) suitable for the user in combination therewith.

3) Additionally, according to the literature [Line Jee Hartmann Rasmussen (2019). Association of Neurocognitive and Physical Function With Walking speed in Midlife, JAMA Netw Open.], a study conducted by researchers from King's College London (KCL) in the UK and Duke University in the US revealed that slower walking speed is associated with faster aging (i.e., slower walking speed is linked to faster aging and increased mortality rates), and that the biological age of people with faster walking speeds may differ by up to 16 years compared to those with slower walking speeds.

In consideration of the foregoing, the guide unit 14 may cause the gait analysis unit 13 to analyze (measure) the user's walking rate and walking speed through the user's gait sound data, and thereafter, based on the analyzed walking rate and walking speed of the user, may provide to the user terminal 20 a third guide information relating to a health walking guide for inducing the user to walk at a recommended walking rate and recommended walking speed corresponding to the user's age, selected from among age-specific recommended walking rates and recommended walking speeds pre-registered by an administrator in a database unit (not shown).

Accordingly, for example, the gait characteristic information analyzed by the gait analysis unit 13 may include not only the first to fourth characteristic information mentioned above but also the fifth characteristic information corresponding to walking speed, among others. Additionally, as an example, the recommended walking speed may be 1.5 meters per second for those in their 30s (e.g., ages 30-39), 1.3 meters per second for those in their 40s (e.g., ages 40-49), and 1.1 meters per second for those in their 50s and 60s (e.g., 50 to 69 years old), and 1 meter per second for those in their 70s and older (e.g., 70 years old and older). This is merely one example to aid in understanding the present invention and is not limited to this, but may be variously set and modified by the administrator.

4) Additionally, the guide unit 14 may provide to the user terminal 20 a fourth guide information related to a running guide that guides the user to walk at a walking rate suitable for moderate-intensity exercise, considering the user's walking rate derived from analyzing the user's gait sound data. In other words, the fourth guide information may refer to information that provides (guides) information regarding the appropriate walking rate for moderate-intensity exercise for the user.

Here, moderate-intensity exercise refers to exercise that maintains the user's heart rate at 50% to 60% of their maximum heart rate (where the maximum heart rate may be derived, for example, as the value of “220 minus age”). Therefore, if only the user's age is known, it is possible to provide the appropriate heart rate and appropriate walking rate to enable the user to walk at a moderate-intensity level. For example, if the user's age is 30, their maximum heart rate is 220 minus 30, which is 190. Therefore, 50% of this is 95, and 60% is 114. Therefore, if the user's age is 30, the appropriate heart rate to enable the user to walk at moderate intensity may be, for example, 95 or higher and 114 or lower (i.e., 95 to 114 beats per minute).

In this case, when providing the fourth guide information, the guide unit 14, in order to provide the user with a more accurate walking rate corresponding to moderate-intensity exercise, may collect information on heart rate and walking rate measured during running performed by multiple experimenters either outdoors or on a treadmill, by using smartwatches (not shown) worn on the wrists of the experimenters. By analyzing the collected information, the guide unit 14 may derive an average walking rate information corresponding to each heart rate. That is, the guide unit 14 may determine the walking rate corresponding to moderate-intensity exercise using the information collected from the smartwatches. Subsequently, the guide unit 14 may extract, as a recommended walking rate for moderate-intensity exercise for the user, the average walking rate information corresponding to the user's appropriate heart rate (for example, between 95 and 114 beats per minute) from the average walking rate information by heart rate, and provide (i.e., provide the fourth guide information including the extracted moderate-intensity walking rate) this to the user terminal 20. For example, if the user's walking rate is determined to be 70 (70 steps per minute) through analysis of the user's gait sound data, and the average walking rate information corresponding to the user's appropriate heart rate is “110 or higher and 130 or lower,” the guide unit 14 may, for example, provide the fourth guide information such as [Your current walking rate is 70, but based on your age, the appropriate walking rate for moderate-intensity exercise (i.e., moderate-intensity exercise walking rate) is 110 or higher and 130 or lower. Please try walking at a faster pace than your usual walking speed to increase your walking rate.] When providing the fourth guide information (i.e., information regarding the moderate-intensity walking rate) to the user terminal 20, the guide unit 14 may provide the moderate-intensity walking rate through auditory guiding in a metronome manner. For instance, right foot sounds may be represented as “thump” and left foot sounds as “tap,” and the beat may be arranged so that the user's walking rate corresponds to 110 beats per minute (BPM). Alternatively, music with a BPM matching the desired walking rate (e.g., 110 BPM) may be provided. By providing the moderate-intensity walking rate as a metronome-style auditory guide, the guide unit 14 may help the user easily reach the recommended exercise intensity (i.e., corresponding to the moderate-intensity walking rate) and facilitates regular exercise.

Additionally, the guide unit 14 may monitor the user's gait sounds in real time (i.e., record the gait sounds in real time and monitor the recorded data) while providing metronome-style auditory guidance related to the fourth guide information, and based on the monitoring results, if one of the user's feet is walking too fast (for example, one foot is too quickly separated from the ground relative to the rhythm) or the gait sound produced by one foot is too quiet relative to the volume of the aforementioned auditory guidance, the guide unit 14 may provide feedback to the user terminal 20 in real time to compensate for this.

Here, the feedback may refer to guidance information that prompts the user to walk in accordance with the rhythm or sound intensity of the auditory guidance, such as “The left foot is leaving the ground too quickly. Please lift the left foot slightly more slowly from the ground,” or “The sound of the left foot is too quiet. Try running so that the sound of your left foot becomes louder.”

FIG. 5 is a diagram illustrating the relationship between the user's walking rate (Cadence) measured by the user terminal 20 and the metabolic equivalent task (MET). Here, the metabolic equivalent task refers to the amount of oxygen required by the body to produce energy while at rest.

Referring to FIG. 5, it may be confirmed that the user's walking rate is proportional to the MET. Considering this, the guide unit 14 may provide the aforementioned feedback to the user based solely on the user's walking rate information derived from the analysis of the user's recorded data (user's gait sound data) when providing the fourth guide information, thereby guiding the user to an appropriate exercise intensity.

### Example of Providing Gait-related Exercise Guidance Information for Patients

The guide unit 14 may provide the following gait-related exercise guidance information (e.g., a fifth guide information to a sixth guide information described below) to the user when the user is a patient.

5) The guide unit 14 may provide a fifth guide information to the user terminal 20 when the user is a patient, measuring the foot contact sound intensity (decibels), foot contact duration (milliseconds, seconds), frequency, walking rate, and foot contact time intervals compared to a normal or healthy foot, and providing personalized feedback. In other words, the guide unit 14 may compare the user's gait pattern information with the reference gait pattern information of normal users of the same age, height, and weight, generate a personalized feedback information as the fifth guide information to guide the user to walk in a gait pattern corresponding to the reference gait pattern information, and provide this information to the user terminal 20. In this case, the guide unit 14 may provide the fifth guide information as auditory guidance (guide) in a metronome manner.

Cerebrovascular diseases have a high prevalence rate in South Korea, with 1.17 million cases reported in 2022, and many cases result in hemiplegia, making walking difficult due to paralysis of one leg. Patients with such conditions as hemiplegia have gait patterns that differ significantly from those of healthy individuals. Therefore, the gait analysis unit 13 may collect not only experimenter data from healthy individuals but also experimenter data from various patients with conditions such as hemiplegia and Parkinson's disease, and use this data to train an artificial intelligence model. Through this training, the gait analysis unit 13 may derive and predefine at least one reference gait pattern information for each physical type for both the normal group and the patient group.

In other words, the gait analysis unit 13 may collect and analyze the experimenter data to analyze (identify, define) the reference gait pattern information for normal individuals and the reference gait pattern information for patients for at least one physical type. Additionally, the gait analysis unit 13 may determine whether the user is a patient or a normal individual using only the gait sound data (or the user's gait characteristic information analyzed from the gait sound data) input into the input unit 12 by applying it as input to the pre-trained artificial intelligence model, even when the user has not pre-entered patient status information. In this case, the guide unit 14 may generate the aforementioned fifth guide information and provide it to the user terminal 20 if the user is determined to be a patient. In other words, the guide unit 14 may enable the patient user to walk efficiently (normal walking) as much as possible through the provision of the fifth guide information.

The guide unit 14 may provide the fifth guide information in the form of an auditory guide using a metronome method to enable the user who is a patient to walk normally as much as possible.

Generally, when left-sided hemiplegia causes poor weight bearing on the left foot, the sound of the left foot touching the ground tends to be relatively shorter and smaller compared to the sound of the right foot touching the ground. Considering this, the guide unit 14 may generate the fifth guide information such that, when the user is determined (confirmed) to be a patient with left hemiplegia and the current gait information of the user derived from the gait analysis unit 13 is exemplarily [the foot contact sound is relatively shorter and smaller in volume compared to the right foot's foot contact sound], the guide unit 14 may generate and provide information such as “Please try walking so that the current left foot contact sound is longer and louder” as the fifth guide information to the user terminal 20.

Additionally, Parkinson's disease has a high prevalence rate, and patients with Parkinson's disease often experience freezing gait, where the initiation of walking is difficult, and shortened stride length, known as shuffling gait. Therefore, the gait analysis unit 13 may collect not only experimenter data from healthy individuals but also experimenter data from various patients (such as those with hemiplegia or Parkinson's disease) and use this data to train (analyze) an artificial intelligence model. Based on this learned (analyzed) information, the guide unit 14 may provide the fifth guide information to users with Parkinson's disease, guiding them to walk with a gait pattern similar to that of normal individuals, thereby enabling users with Parkinson's disease to walk more efficiently.

6) Additionally, patients with various conditions such as hemiplegia, paraplegia, cerebral palsy, and Parkinson's disease have gait patterns that differ from those of healthy individuals, as mentioned earlier. Therefore, the guide unit 14 may apply the user's gait sound data to a pre-trained artificial intelligence model. If the user is determined to be a patient, the guide unit 14 may provide a sixth guide information related to hospital visit guidance to the user terminal 20, enabling the user to receive sound analysis and guidance regarding walking under the supervision of a doctor or physical therapist at the hospital.

In this way, the guide unit 14 may provide customized gait-related exercise guidance information to users with various diseases or without any diseases, based on the user's gait information. In other words, the user of the user terminal 20 in the present invention may be a user (patient) with any type of paralysis (e.g., hemiplegia, paraplegia, cerebral palsy, etc.), a normal user without any disease, or an athlete, etc. The guide unit 14 may provide gait-related exercise guide information that guides the user to walk normally, based on the user's gait information, as customized guide information (gait-related exercise guide information). The guide unit 14 may provide the gait-related exercise guide information, particularly the second guide information to the fifth guide information mentioned above, to the user in the form of metronome-style auditory guidance.

In this case, the guide unit 14 may determine whether the beat of the metronome provided to the user matches the beat of the sound produced by the user's walking, or the degree of match (matching rate), and provide (feedback) a score for the determined matching rate to the user terminal 20. Here, the score for the matching rate may be derived and provided such that the higher the matching rate between the rhythm of the user's gait sound and the metronome's rhythm, the higher the score, and the lower the matching rate, the lower the score. In other words, the guide unit 14 may provide feedback such as a score when the user matches the rhythm well.

Additionally, the guide unit 14 may provide the gait-related exercise guidance information through metronome-style auditory guidance, and the gait-related exercise guidance information (particularly content that guides the user to walk in sync with the metronome's rhythm) may be: i) for example, as shown in FIG. 6(a), displayed on a first type of display device such as a TV, a monitor, or ii) as shown in FIG. 6(b), a beam projector (mini beam projector), a head-up display (HUD), or other second-type display devices to project beam information corresponding to the gait-related exercise guide information onto the front via the second-type display devices, or iii) as shown in FIG. 6(c), VR (Virtual Reality) glasses, smart glasses (wearable glasses), and other third-type display devices, and project virtual three-dimensional information (i.e., virtual three-dimensional gait exercise guide information) corresponding to the gait exercise guide information onto the front through the third-type display devices.

That is, if the guide unit 14 provides the gait-related exercise guide information to the user's smartphone (user terminal 20) when the user is walking while viewing the gait-related exercise guide information on the smartphone, this could be difficult and dangerous for the user. To address this issue, the guide unit 14 may provide the gait-related exercise guide information by linking it with a first type of display device such as a TV and displaying it on the screen, thereby visualizing the gait-related exercise guide information more clearly and providing it to the user in a safer and more convenient manner. Additionally, the guide unit 14 may provide gait-related exercise guide information by linking with a first type of display device as well as a running machine device (i.e., it may be used while using a treadmill), thereby enabling the user to perform walking in the correct posture on the treadmill while viewing the gait-related exercise guide information displayed on the screen of the first type of display device in a metronome-like manner.

Additionally, the guide unit 14 may provide gait-related exercise guidance information to the second type of display device and the third type of display device, thereby enabling the provision of gait-related exercise guidance information to the user in environments such as augmented reality or virtual reality. In particular, when the guide unit 14 provides gait-related exercise guidance information to the second type of display device and the third type of display device, it may provide the user with an image icon (image) of a foot shape corresponding to the right foot and left foot, which are appropriate for walking, by projecting (illuminating) the image icon toward the ground (floor) where the user is located, thereby enabling the user to walk in accordance with the stride length corresponding to the right foot and left foot indicated by the image icon.

In other words, the guide unit 14 may be implemented using augmented reality when providing gait-related exercise guide information to the user, and may be used in conjunction with a mini beam projector that projects an appropriate stride length onto the floor in the form of a foot shape. Additionally, the guide unit 14 may provide gait-related exercise guide information to the user by combining it with VR glasses or future smart glasses, integrating various elements that guide the user toward the path, and providing both auditory and visual guides, as well as feedback information.

The guide unit 14 may provide gait-related exercise guidance information to the user based on at least one of augmented reality (AR), virtual reality (VR), mixed reality (MR), and metaverse.

The control unit 15 may control the operation of each unit (i.e., recording control unit, input unit, gait analysis unit, and guide unit) within the device 10. Additionally, the control unit 15 may control the operation (such as a screen display activation) of the user terminal 20 connected to the device 10 via the network 5.

As described above, the device 10 may acquire gait sound data recorded using a user terminal 20 that is a smartphone carried by a user during walking, perform analysis of the user's gait based on the acquired gait sound data, and provide customized gait-related exercise guide information to the user based on the gait information obtained from the analysis.

The device 10 may easily analyze the user's gait and provide exercise guidance (i.e., gait-related exercise guidance information) through a smartphone.

The device 10 may be designed for normal individuals, and when the user is a normal individual, it may analyze the user's walking by analyzing the sound (recorded gait sounds) of the user's walking, and based on this, provide the user with guide information (gait-related exercise guide information) regarding issues in the user's walking and appropriate walking or running methods.

Additionally, the device 10 may provide the user with gait-related exercise guidance information when the user is a normal individual, including: ii) information on which of the user's two legs is shorter, information on which of the two legs has insufficient force, information on whether the user is walking at the recommended appropriate speed, information on whether the user is running at the recommended appropriate speed, content information on stretching exercises for the shorter leg, and speed guide information to encourage the user to walk or run at the recommended appropriate speed, and provide the user with gait-related exercise guidance information.

Additionally, the device 10 may provide, when the user is normal, iii) gait-related exercise guide information based on the user's age, including normal gait development information for children (e.g., between 6 and 12 years old), exercise methods for functional improvement for adolescents and adults (e.g., between 13 and 64 years old), and recommended walking speeds for health promotion for elderly users (e.g., 65 years or older). In this case, the gait-related exercise guide information provided to the elderly may be generated based on research findings indicating that the slower the walking speed, the higher the mortality rate, as mentioned earlier, and may refer to the third guide information (i.e., the third guide information regarding a health walking guide that encourages users to walk at the recommended walking rate and speed corresponding to their age).

The device 10 may provide the user with metronome-style gait-related exercise guidance information that guides the user's walking to be closer to normal walking (i.e., normal gait pattern information) when the user is a patient, particularly when the patient has paralysis in one leg or both legs are impaired. As a specific example, the device 10 may provide the user with gait-related exercise guidance information that includes information (i.e., information that allows confirmation of the user's walking development level) enabling confirmation of whether the user's walking is developing well compared to the gait pattern information of a normal child, when the user is a pediatric patient with developmental delay or paralysis. Additionally, the device 10 may provide the user with gait-related exercise guide information that includes information on the walking speed of the unaffected (healthy) leg or the walking speed of a normal person when the user is a patient with hemiplegia or paraplegia.

The device 10 may collect data (experimenter data) from normal individuals and patient groups for the provision of the service and perform analysis of the user's current gait information based on AI. In this case, the device 10 may collect experimenter data from normal individuals and patients separately, and in this case, it may collect variable information such as age, height, body weight, and leg length, and use this information to train the AI model. Through this training, the device 10 may obtain and define average data (i.e., at least one reference gait pattern information for each physical type in the normal group and patient group) for the experimenters. Additionally, since walking is an endurance-related activity and patterns (gait patterns) change over time, the device 10 may continuously collect and train experimenter data adjusted for age changes, thereby enabling calibration of the aforementioned average data.

When a user accesses the app or program and inputs (uploads) their gait sound data, the device 10 may respond by generating gait-related exercise guide information tailored to the user to help them walk normally, and provide this information in the form of metronome-based auditory guidance. When providing gait-related exercise guidance information to the user, the device 10 may provide such information based on default auditory guidance, or based on personalized metronome-style auditory guidance, thereby guiding the user toward more comfortable and desirable normal walking.

Additionally, the device 10 may provide gait-related exercise guidance information in various forms, such as visual or tactile, by linking it with virtual reality, augmented reality, etc. That is, when providing gait-related exercise guidance information, the device 10 may combine virtual reality, augmented reality, etc., to display the shape of the foot in the user's field of view, thereby providing not only visual guidance but also tactile guidance such as vibration. The device 10 may provide gait-related motion guidance information based on augmented reality, virtual reality, mixed reality, or metaverse by linking with additional equipment (e.g., VR devices, smart glasses, beam projectors, etc.).

Conventional walking guide technologies (e.g., conventional walking guide applications using auditory cues) require measurement equipment (e.g., separate measurement devices such as IMUs) and, in particular, necessarily require the installation of recording devices on both feet or ankles, resulting in a somewhat complex measurement process that may be cumbersome for users. In contrast, the present device 10 may provide gait-related guidance using only the recording function of the user's smartphone without requiring such measurement equipment, enabling users to easily receive information about their current walking status or gait-related exercise guidance tailored to their needs anytime, anywhere, without being restricted by location.

Additionally, conventional walking guide technologies do not distinguish between right and left foot movements. In contrast, the present device 10 enables users to distinguish between the right and left feet during recording by allowing them to decide, through the app, whether to start with the right foot or the left foot (i.e., by selecting the information regarding the first step of walking during recording through the recording progress screen), thereby enabling recording of both feet. In other words, the device 10 may enable clear distinction between the two feet during walking (i.e., clear recording of the sounds made by the two feet during walking) by allowing the user to select the information regarding the first starting foot, so that when the recording is performed, the two feet alternately touch the ground as walking progresses. Additionally, the device 10 may enable a real-time verification during the recording process to confirm whether the right and left feet are being recorded distinctly, and allows the user to perform re-recording if necessary. FIG. 3 illustrates an example of recorded data showing the sounds produced during walking when the user performs walking indoors barefoot using the Galaxy user terminal 20.

Furthermore, the device 10 may provide gait-related exercise guidance information to the user in the form of customized, regular metronome-based auditory guidance to encourage normal walking, and may additionally incorporate music or noise (pink noise) into the guidance.

Furthermore, conventional walking guide technologies often have very limited data and are designed for use by patients with specific conditions (e.g., Parkinson's disease). In contrast, the present device 10 does not require separate measurement devices, enabling it to be used by a wide range of users, including adults, the elderly, patients with various types of leg paralysis, patients with various diseases, athletes, and others, without being limited to patients with specific conditions. Additionally, the present device 10 may provide real-time gait-related feedback to the user based on the user's current walking status or gait-related exercise guide information.

Hereinafter, we will briefly review the operational flow of the present invention based on the detailed description provided above.

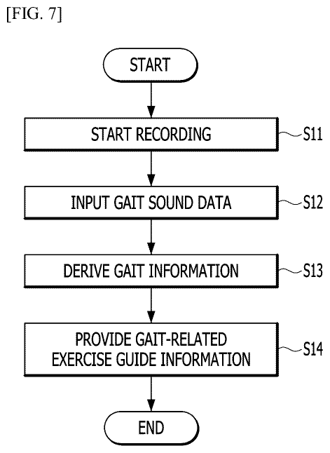

FIG. 7 is a flowchart illustrating the gait analysis method according to an embodiment of the present invention.

The gait analysis method shown in FIG. 7 may be performed by the device 10 described above. Therefore, even if the contents are omitted, the descriptions of the device 10 apply equally to the description of the gait analysis method.

Referring to FIG. 7, in step S11, the recording control unit may control the recording of sounds made by the user during walking.

Next, in step S12, the input unit may receive gait sound data, which is data recorded of the user's gait sounds.

Next, in step S13, the gait analysis unit 13 may derive the user's current gait information through analysis of the gait sound data input in step S12.

Next, in step S14, the guide unit 14 may generate and provide gait-related exercise guide information based on the gait information derived in step S13.

As described above, steps S11 to S14 may be further divided into additional steps or combined into fewer steps depending on the implementation example of the present invention. Additionally, some steps may be omitted as necessary, and the order of steps may be changed.

The gait analysis method according to an embodiment of the present invention may be implemented in the form of program instructions that may be executed by various computer means and recorded on a computer-readable medium. The computer-readable medium may include program instructions, data files, data structures, etc., alone or in combination. The program instructions recorded on the medium may be specially designed and configured for the present invention or may be known to those skilled in the art of computer software. Examples of computer-readable recording media include magnetic media such as hard disks, floppy disks, and magnetic tapes, optical media such as CD-ROMs and DVDs, magneto-optical media such as floptical disks, and hardware devices specially configured to store and execute program instructions, such as ROM (read-only memory), RAM (random access memory), and flash memory. Examples of program instructions include machine code generated by a compiler, as well as high-level language code that may be executed by a computer using an interpreter or similar mechanism. The aforementioned hardware devices may be configured to operate as one or more software modules to perform the operations of the present invention, and vice versa.

Additionally, the aforementioned gait analysis method may be implemented in the form of a computer program or application executed by a computer stored on a recording medium.

According to the embodiments of the present invention described above, by providing a gait analysis device and method, gait sound data recorded using a user terminal (e.g., a smartphone) may be easily acquired, analysis of the user's gait may be performed based on the acquired gait sound data, and a customized gait-related exercise guide information may be provided to the user based on the analysis results, thereby assisting the user in walking with a more proper posture.

According to the aforementioned embodiments of the present invention, by providing a gait analysis device and method, even users who do not have dedicated gait detection measurement equipment (e.g., IMU) may easily perform analysis of their gait, and anyone who wishes to analyze their gait including patients with specific diseases (e.g., leg paralysis, Parkinson's disease, cerebral palsy, etc.) as well as various general users (e.g., adults, children, athletes, etc.) who wish to analyze their walking may receive analysis information about their gait (i.e., the user's current gait information), and the user may receive a customized gait-related exercise guidance information that guides the user to change their gait to a more correct posture based on their current gait information.

However, the effects obtainable from the present invention are not limited to the effects described above, and other effects may exist.

The above description of the present invention is for illustrative purposes only, and those skilled in the art will understand that the technical concepts and essential features of the present invention may be easily modified into other specific forms without changing the technical concepts or essential features of the present invention. Therefore, the embodiments described above should be understood as illustrative and not limiting in all respects. For example, each component described as a single unit may be implemented in a distributed manner, and similarly, components described as distributed may be implemented in a combined form.

The scope of the present invention is defined by the patent claims set forth below, rather than by the detailed description above, and all modifications or variations derived from the meaning, scope, and equivalent concepts of the patent claims are included within the scope of the present invention.

What is claimed is:

1. A gait analysis device comprising:
an input unit configured to receive a gait sound data of a user;
a gait analysis unit configured to derive a gait information of the user through analysis of the gait sound data; and
a recording control unit configured for the user to control recording of sounds during walking using a smartphone as a user terminal,
wherein the recording control unit is configured to:
display a recording progress screen on a screen of the user terminal, wherein the recording progress screen includes a start foot selection button and a recording start button for selecting information about a first starting foot of the user's walking when recording is being performed,
guide, thereafter, the user to select one of a left button and a right button as a button corresponding to the first start foot when the user performs the recording on the a recording progress screen, wherein the left button is included within the start foot selection button and is configured to be selected when the first start foot is a left foot, wherein the right button is included within the start foot selection button and is configured to be selected when the first start foot is a right foot,
guide the user, when a button of the left button and the right button is selected, to perform walking with a foot corresponding to the selected button as the first starting foot after the user clicks the recording start button,
display in real time on the screen of the user terminal, when the user clicks the recording start button, a waveform data corresponding to gait sounds recorded by the user terminal's recorder as the user walks, and
guide the user to click a re-record button displayed in an area of the screen, when the user confirms that the waveform data displayed in real time does not distinguish between a waveform data of the left foot and a waveform data of the right foot, thereby initiating re-recording of the gait sounds.

2. The gait analysis device of claim 1, wherein the input unit is configured to receive a recording data of the gait sounds recorded through the recording control unit as the gait sound data.

3. The gait analysis device of claim 1, wherein the gait analysis unit is configured to derive the gait information by comparing a user gait pattern information derived from analyzing the gait sound data with a reference gait pattern information predefined through analyzing a gait sound data of a plurality of experimenters previously collected.

4. The gait analysis device of claim 1, wherein the gait analysis device further includes a guide unit that is configured to generate and provide a gait-related exercise guide information based on the gait information.

5. A gait analysis method capable of being performed by the gait analysis device of claim 1, comprising:
receiving the gait sound data of the user in the input unit; and
deriving the gait information of the user through analysis of the gait sound data in a gait analysis unit.
